(19) United States
(12) Plant Patent Application Publication
(10) Pub. No.: US 2009/0151031 P1
(43) Pub. Date: Jun. 11, 2009

(54) Cherry Tree Named 'Maddison'

(50) ;

(76) Inventor: Ed Gooch, Malaga, WA (US)

Correspondence Address: STRATTON BALLEW, 213 S 12TH AVE, YAKIMA, WA, 98902, US

(21) Appl. No.: 11/999,960

(22) Filed: Dec. 7, 2007

(51) Int. Cl. A01H 5/00

(52) U.S. Cl. PLT/181

(57) ABSTRACT

‘Maddison’ is a new and distinct cherry tree notable for its early ripening, large size, and dark color as compared to ‘Bing.’

### CROSS-REFERENCE TO RELATED APPLICATIONS

None

### STATEMENT REGARDING FEDERALLY SPONSORED RESEARCH OR DEVELOPMENT

None

### GENUS AND SPECIES

Prunus avium

### VARIETY DENOMINATION

‘Maddison’

### BACKGROUND AND SUMMARY OF THE VARIETY

The new cherry variety ‘Maddison’ originated as a chance seedling of ‘Bing’ (not patented). The seedling was found in a commercial orchard near Malaga, Wash., in 2004. The seedling was distinguishable from ‘Bing’ by its early ripening, large size, and dark color. Trees were propagated from the seedling by chip budding onto ‘Mazzard’ rootstock, to determine whether the desired characteristics of the chance seedling would carry through to asexually propagated progeny. It has been found that the characteristics of early ripening, large size, and dark color, as well as other characteristics of the tree and its fruit, were reproduced through asexual propagation and have remained stable through successive generations.

‘Maddison’ is a new and distinct cherry tree notable for its early ripening, large size, and dark color as compared to ‘Bing.’

### BRIEF DESCRIPTION OF THE PHOTOGRAPHS

FIG. 1 shows the fruit and leaves of the new variety;

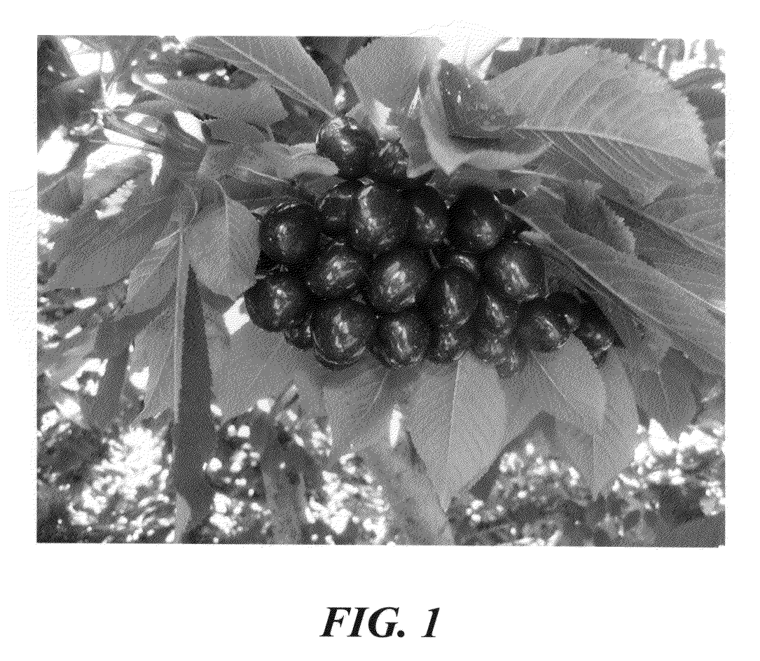

FIG. 2 shows the blossoms of the new variety; and

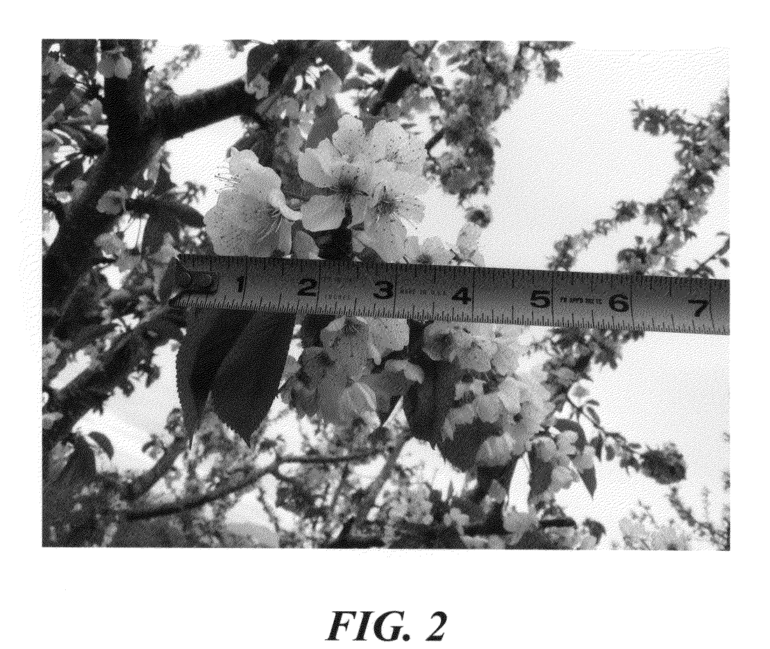

FIG. 3 shows the tree of the new variety.

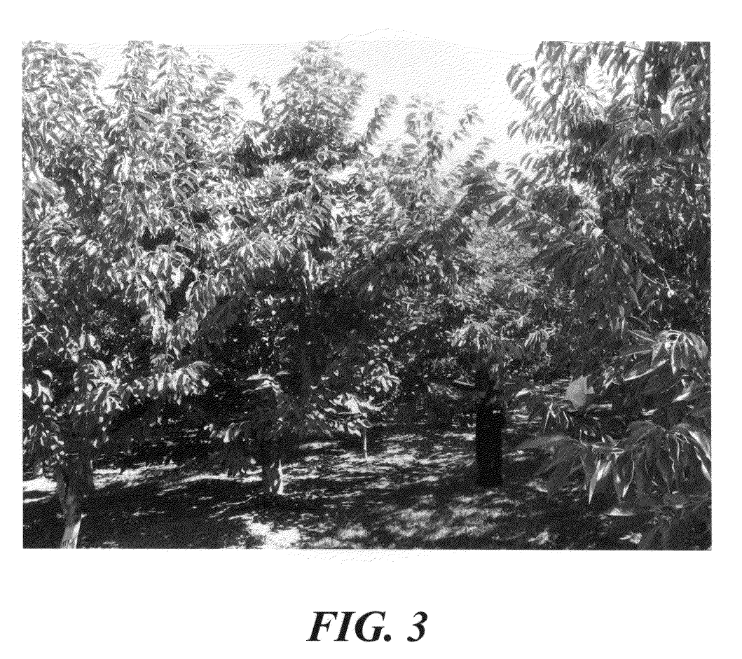

The colors of this illustration may vary with lighting conditions. Color characteristics of this new variety should therefore be determined with reference to the observations described herein, rather than from these illustrations alone.

### DETAILED BOTANICAL DESCRIPTION

The following detailed botanical description is based on observations of the nine year old parent tree, made during the 2007 growing season at Malaga, Wash. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree:
   Size.—Height 5 m, spread 4.4 m.
   Vigor.—Vigorous, about 70 cm per year.
   Branching habit.—Spreading to upright.
   Density.—Dense.
   Hardiness.—Hardy in area tested.
   Productivity.—95 kg (2007 growing season).
   Bearing.—Annual.
   Trunk.—Diameter 22.8 cm at 15 cm above soil; Bark somewhat rough, gray 201D; Trunk lenticel width 2 mm, length varies, orange-white 159A.
   Branch.—Diameter 4 cm at 150 cm from trunk, length 1.4 m, crotch angle 20° to 30°, texture smooth, greyed-orange 175A, branch lenticel width 2 mm, length varies, quantity 6 per square inch.
Leaves:
   Size.—Length 18.4 cm, width 8.2 cm.
   Shape.—Elliptic, base equilateral, apex acuminate, recurved inward.
   Margin.—Serrate.
   Color.—Upper surface green 137A, lower surface green 137D.
   Midvein.—Width 3 mm, upper surface yellow-green 145C, lower surface green 142C.
   Petiole.—Length 44 mm, diameter 1.5 mm; upper surface yellow-green 146C, lower surface green 142C.
   Glands.—Quantity 2 per leaf; oval; length 3 mm, width 2 mm; yellow-green 146C.
Flowers:
   Buds.—Quantity 10 per spur; length 1.3 cm, width 0.5 cm; yellow-green N144D.
   Bloom date.—First bloom April 6; full bloom date April 13 (2007 growing season, Malaga, Wash.).
Fruit:
   Size.—Apical diameter 28 mm, axial diameter 25 mm.
   Shape.—Heart shaped, symmetrical.
   Stem.—Length 4.4 cm, diameter 2 mm; green 142C.
   Cavity.—Depth 4 mm, diameter 9 mm.
   Skin.—Thickness 0.5 mm, smooth, tenacious to flesh, no tendency to crack noted; red purple 59A.
   Flesh.—Firm, juicy; red purple 59A.
   Stone.—Length 13 mm, width 7 mm; oval; flesh not tenacious to stone; greyed-orange 163D.
   Maturity date.—Eating ripe on June 20; date of first and last picking June 20 (2007 growing season, Malaga, Wash.).

What is claimed is:

1. A new and distinct cherry tree as shown and described herein.
